(19) United States
(12) Patent Application Publication
(10) Pub. No.: US 2022/0369793 A1
(43) Pub. Date: Nov. 24, 2022

(54) MASK APPARATUS

(71) Applicant: Peach Tech, Seoul (KR)

(72) Inventor: Hae Sung PARK, Seoul (KR)

(73) Assignee: Peach Tech, Seoul (KR)

(21) Appl. No.: 17/774,164

(22) PCT Filed: Nov. 5, 2020
(86) PCT No.: PCT/KR2020/015411

§ 371 Date: May 4, 2022

(30) Foreign Application Priority Data
Nov. 5, 2019 (KR) 10-2019-0140149
Jul. 10, 2020 (KR) 10-2020-0085335

(51) Int. Cl. A45D 44/00

(52) U.S. Cl. CPC A45D 44/002; A45D 2200/205

(57) ABSTRACT

An embodiment discloses a mask apparatus including a cover mask having a curvature corresponding to a shape of a face of a user and a plurality of light-emitting modules arranged in the cover mask to irradiate light to the face of the user.

### TECHNICAL FIELD

An embodiment relates to a mask apparatus that irradiates light to a face.

### BACKGROUND ART

With recent increasing interest in beauty, the popularity of LED masks that irradiate light to a face is high.

Generally, an LED mask is manufactured by mounting an SMD-type LED package (including a red LED, a near-infrared LED, etc.) on an FPCB and inserting the same into a plastic case. Thus, light is irradiated from an inner side of the case apart from a user's face by a predetermined gap.

As a result, light that is significantly less than the amount of light required for skin beauty is irradiated, such that a beauty effect hardly appears. Moreover, the LED mask may dry out the skin, resulting in damage to the skin.

### DISCLOSURE

### Technical Problem

An embodiment provides a mask apparatus capable of irradiating light of an amount required for skin beauty.

Moreover, an embodiment provides a mask apparatus having an improved massage effect by preventing skin dryness.

Problems to be solved in embodiments are not limited thereto, and may include objects or effects that may be understood from solutions to the problems or embodiments described below.

### Technical Solution

According to an aspect of the present disclosure, a mask apparatus includes a cover mask having a curvature corresponding to a shape of a face of a user and a plurality of light-emitting modules arranged in the cover mask to irradiate light to the face of the user.

The cover mask may include a plurality of mounting grooves mounted on an inner surface thereof, the plurality of light-emitting modules may be arranged in the plurality of mounting grooves, and the light-emitting module may include a circuit board arranged in each of the plurality of grooves and a plurality of light-emitting elements arranged in the circuit board.

The light-emitting module may include an elastic pad filled in the mounting groove to cover the light-emitting element, and the elastic pad may protrude from the inner surface of the cover mask such that a top surface of the elastic pad contacts the face of the user.

The mask apparatus may further include an elastic pad attached to the inner surface of the cover mask to cover the mounting groove.

The cover mask may include a plurality of opening portions formed in a region in which the light-emitting module is arranged, and a frame portion arranged between the plurality of opening portions, and the light-emitting module may be attached to the frame portion.

The region in which the light-emitting module is arranged may include a region corresponding to a forehead, a cheek, and a nose of the user.

The frame portion may have the curvature corresponding to the shape of the face of the user.

The mask apparatus may include a case arranged on an outer side of the cover mask.

### Advantageous Effects

According to an embodiment, as a light-emitting element is arranged on a mask manufactured according to a shape of a face of a user and an elastic pad closely contacts the face, such that moisture evaporation is suppressed and thus the face is prevented from drying out. Therefore, light having a power sufficient to improve the skin may be irradiated.

Various and useful advantages and effects of the present invention are not limited to the foregoing description, and may be more easily understood in a process of describing detailed embodiments of the present invention.

### DESCRIPTION OF DRAWINGS

FIG. 1 is a conceptual diagram of a mask apparatus according to a first embodiment of the present disclosure.

FIG. 2 is an enlarged view of a portion A of FIG. 1.

FIG. 3 is a view showing a state in which a light source module is in close contact with a face of a user.

FIG. 4 shows a conventional mask apparatus.

FIG. 5 is a conceptual diagram of a mask apparatus according to a second embodiment of the present disclosure.

FIG. 6 is a view showing a state in which a light source module is in close contact with a silicon pad placed on a face of a user.

FIG. 7 is a plane view of a cover mask according to the present disclosure.

FIG. 8 is an enlarged view of a region in which a light source module is mounted.

FIG. 9 is a conceptual diagram of a light source module.

FIG. 10 is a plane view showing a state in which a light source module is mounted on a cover mask.

FIG. 11 is an enlarged view of a mounting groove formed in a region in which a light source module is mounted.

FIG. 12 shows a state where a light source module is arranged in a mounting groove.

FIG. 13 shows an apparatus for manufacturing a mask.

FIG. 14 is a conceptual diagram of a mask module according to a third embodiment of the present disclosure.

FIG. 15 is a conceptual diagram of a mask module according to a third embodiment of the present disclosure.

FIG. 16 is a conceptual diagram of a mask module according to a fourth embodiment of the present disclosure.

FIG. 17 shows that an elastic pad is in close contact with a face to prevent moisture from being evaporated.

FIG. 18 shows a state in which sweat is evaporated through a gap between a conventional LED mask and a face.

FIG. 19 shows arrangement of a plurality of light-emitting elements.

FIG. 20 shows a mask module according to a fifth embodiment of the present disclosure.

FIG. 21 shows a mask module according to a sixth embodiment of the present disclosure.

FIG. 22 is a flowchart of a method for manufacturing a mask module, according to a third embodiment of the present disclosure.

FIG. 23 shows a process of obtaining facial shape information.

FIG. 24 shows an apparatus for manufacturing a mask.

FIGS. 25 and 26 show a process of manufacturing an elastic pad by using a mold.

FIG. 27 is a conceptual diagram of a manufactured mask module.

### BEST MODE

Various changes may be made to the present disclosure and the present disclosure may have various embodiments which will be illustrated in the drawings and described in detail in the detailed description. However, such a description is not construed as limited to specified embodiments, and include all changes, equivalents, or substitutes included in the spirit and technical scope of the present invention.

Although ordinal numbers such as “first”, “second”, and so forth will be used to describe various components of the present invention, those components are not limited by the terms. These terms may be used for the purpose of distinguishing one component from another component. For example, a second component may also be named as a first component without departing from the right scope of the present invention, and similarly, the first component may also be named as the second component. The term “and/or” used herein includes any and all combinations of one or more of the associated listed items.

When a component is referred to as being “connected” or “accessed” to or by any other component, it should be understood that the component may be directly connected or accessed by the other component, but another new component may also be interposed between them. Contrarily, when a component is referred to as being “directly connected” or “directly joined” to or by any other component, it should be understood that there is no component between the component and the other component.

The terms used in the present application are used for the purpose of describing particular exemplary embodiments only and are not intended to be limiting. Singular forms include plural forms unless apparently indicated otherwise contextually. It will be further understood that the terms “comprises” and/or “has,” when used in this application, specify the presence of a stated feature, number, step, operation, component, element, or combination thereof, but do not preclude the presence or addition of one or more other features, numbers, steps, operations, components, elements, or combinations thereof.

All of the terms used herein including technical or scientific terms have the same meanings as those generally understood by an ordinary skilled person in the related art unless they are defined other. The terms defined in a generally used dictionary should be interpreted as having meanings that are the same as or similar with the contextual meanings of the relevant technology and should not be interpreted as having ideal or exaggerated meanings unless they are clearly defined in the embodiments.

Hereinafter, an embodiment will be described in detail with reference to the accompanying drawings, and regardless of figure symbols, the same component or corresponding components will be given the same reference numeral and a redundant description will not be provided.

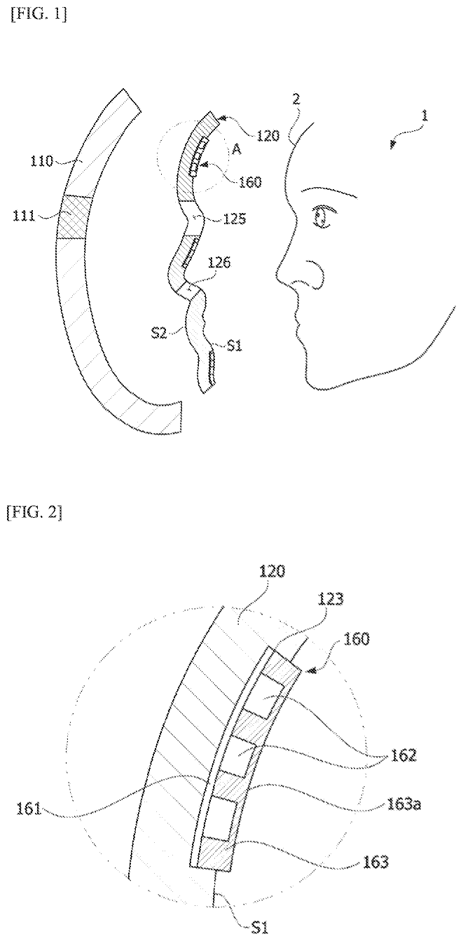

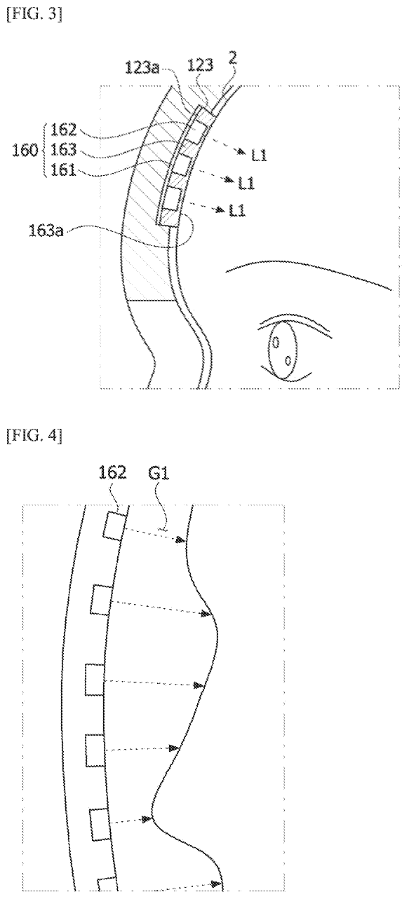

FIG. 1 is a conceptual diagram of a mask apparatus according to a first embodiment of the present disclosure, FIG. 2 is an enlarged view of a portion A of FIG. 1, FIG. 3 is a view showing a state in which a light source module is in close contact with a face of a user, and FIG. 4 shows a conventional mask apparatus.

Referring to FIGS. 1 and 2, a mask apparatus according to an embodiment may include a cover mask 120 having a curvature corresponding to a shape of a face of a user, and a plurality of light-emitting modules 160 arranged in the cover mask 120 to irradiate light to the face of the user.

The cover mask 120 may be manufactured with, but not necessarily limited to, a plastic material, and various materials having a predetermined strength may also be selected. Both an inner surface S1 and an outer surface S2 of the cover mask 120 may have the same shape as the shape of the face 2 of the user 1. The cover mask 120 may be manufactured using a 3D printer to have the same shape as the shape of the face 2 of the user 1. However, the present disclosure is not limited thereto, such that various manufacturing techniques capable of manufacturing a mask according to a shape of a face of a user may be applied without any limitation.

The cover mask 120 may include exposure holes 125 and 126 that expose an eye and a nose of the user. In addition, although not shown in the drawing, the cover mask 120 may further include an exposure hole that exposes a mouth of the user. However, without being necessarily limited thereto, some exposure hole may not be formed when necessary.

A plurality of mounting grooves 123 may be formed in the inner surface S1 of the cover mask 120, and the plurality of light-emitting modules 160 may be arranged inside the mounting groove 123. The light-emitting module 160 may include a plurality of light-emitting elements 162 arranged on a circuit board 161 and an elastic pad 163 filled in the mounting groove 123 to cover the plurality of light-emitting elements 162. The plurality of light-emitting elements 162 may irradiate light toward the face of the user. The plurality of light-emitting elements 162 may irradiate light in a wavelength band that is useful for the skin. For example, the plurality of light-emitting elements 162 may have an output wavelength of at least one of 390 nm, 450 nm, 580 nm, 630 nm, and 700 nm. The plurality of light-emitting elements 162 may have different output wavelengths. The plurality of light-emitting elements 162 may emit light in a near-infrared wavelength band of 600 nm to 940 nm.

The plurality of light-emitting elements 162 may be light-emitting diodes, organic light-emitting diodes, or laser diodes. That is, the plurality of light-emitting elements 162 are not specially limited as long as they may irradiate light helpful to improve a facial skin tone for brightening the same or maintain facial elasticity.

The elastic pad 163 may be filled in the mounting groove 123 to cover the plurality of light-emitting elements 162. The elastic pad 163 may be manufactured by filling and hardening silicon-based resin in the mounting groove 123. However, the type of resin forming the elastic pad 163 is not specially limited.

The elastic pad 163 may be formed such that a top surface 163a protrudes from the mounting groove 123. With this structure, the top surface 163a of the elastic pad 163 may closely contact the face of the user and the other part without the elastic pad may be separated from the face of the user by a predetermined gap. Thus, the inner surface S1 of the cover mask 120 may be separated from the face of the user by the predetermined gap.

However, the present disclosure is not limited thereto, such that the top surface 163a of the elastic pad 163 may be formed not to protrude from the mounting groove 123 or may protrude from the mounting groove 123 to form a layer covering the inner surface of the cover mask as a whole. The elastic pad 163 may be manufactured in advance in manufacturing of a light-emitting module and may be inserted into the mounting groove 123.

The case 110 may be coupled to the cover mask 120 to protect an exterior of a mask module. The case 110 may have formed therein a window 111 through which the user may observe an outside.

The cover mask 120, when the case 110 is attached thereto, may be manufactured to merely have a functional part, thereby simplifying a manufacturing process. When the cover mask 120 is an outermost cover, the cover mask 120 needs to be finished separately.

That is, the cover mask 120 is manufactured in a customized shape depending on a user, but the case 110 may be manufactured in a standardized single size. The case 110 is manufactured to have a shape forming an exterior, but the cover mask 120 may be manufactured to correspond to the shape of the face of the user. Thus, a gap may be formed between the case 110 and the cover mask 120.

The case 110 or the cover mask 120 may accommodate required parts of a mask module, e.g., a control module, a circuit module, a power module, etc.

A fixing unit (not shown) connected to the cover mask 120 or the case 110 to fix the mask module to the head of the user may be further included. The fixing unit may be of a band type, but may not be particularly limited as long as it is capable of fixing the cover mask 120 or the case 110 to the head of the user.

Referring to FIG. 3, the cover mask 120 has a shape corresponding to the shape of the face 2 of the user 1, such that the light-emitting module 160 mounted on the cover mask 120 may also be arranged according to the shape of the face of the user. More specifically, when a bottom surface 123a of the mounting groove 123 is formed according to the shape of the face of the user, a top surface of the light-emitting module 160 arranged thereon may also be arranged according to the shape of the face of the user. Thus, light L1 emitted from the light-emitting module 160 may be incident substantially perpendicularly to the face of the user. As a result, light injection efficiency may be improved by minimizing light reflection from the skin.

The top surface 163a of the elastic pad 163 may be formed to protrude from the inner surface of the cover mask 120. Thus, when the cover mask 120 is used, the elastic pad 163 may closely contact the face of the user. Hence, moisture such as sweat generated by light irradiation may not be discharged to the outside, thus maintaining a moisturizing effect. Moreover, sweat may not be easily discharged from a part closely contacting the elastic pad 163, thereby preventing the skin from drying out. Consequently, by sufficiently increasing the intensity of light, a beauty effect may be maximized.

Referring to FIG. 4, a conventional LED mask is manufactured to have a predetermined curve differently from a shape of a face of a user, such that the light-emitting element 162 is not arranged along a curve of the face. As a result, light is not incident perpendicularly to the skin, degrading injection efficiency.

Moreover, a gap G1 exists between an inner surface of the mask and the face, such that moisture (sweat) and heat generated by light irradiation are easily discharged to the outside, making the skin dry. When the power of light is increased in a dry state of the skin, the skin may be even damaged. Consequently, in a conventional structure, the effect was not sufficient because the power of light may not be raised high.

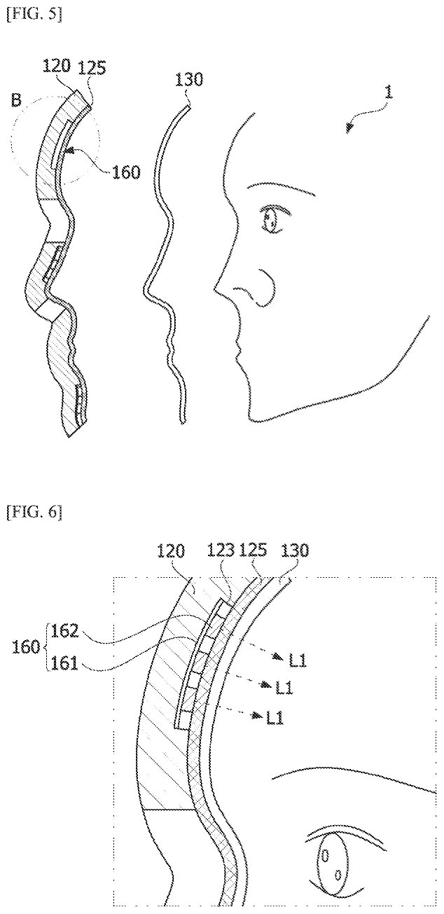

FIG. 5 is a conceptual diagram of a mask apparatus according to another embodiment of the present disclosure, and FIG. 6 is a view showing a state in which a light source module is in close contact with a silicon pad placed on a face of a user.

Referring to FIGS. 5 and 6, a mask apparatus according to an embodiment may include the cover mask 120 having a curvature corresponding to the shape of the face of the user, the plurality of light-emitting modules 160 arranged in the cover mask 120 to irradiate light to the face of the user, and a cover film 125 covering an inner surface of the cover mask 120.

According to an embodiment, the cover film 125 covers the inner surface of the cover mask 120 as a whole, such that the plurality of light-emitting modules 160 may be fixed between the cover mask 120 and the cover film 125. The cover film 125 may be formed by disposing the plurality of light-emitting modules 160 on the cover mask 120 and applying resin to a back surface of the cover mask 120 as a whole. Alternatively, the separately manufactured cover film 125 may be attached to the back surface of the cover mask 120. The cover film may be formed on a part covering the plurality of light-emitting modules 160, instead of being formed on the back surface of the cover mask 120 as a whole.

When the user wears the cover mask 120 after placing the separate silicon pad 130 on the face, the cover film 125 may closely contact the silicon pad 130. In this case, the cover film 125 and the silicon pad 130 may have the same composition, but without being limited thereto, they may have different compositions.

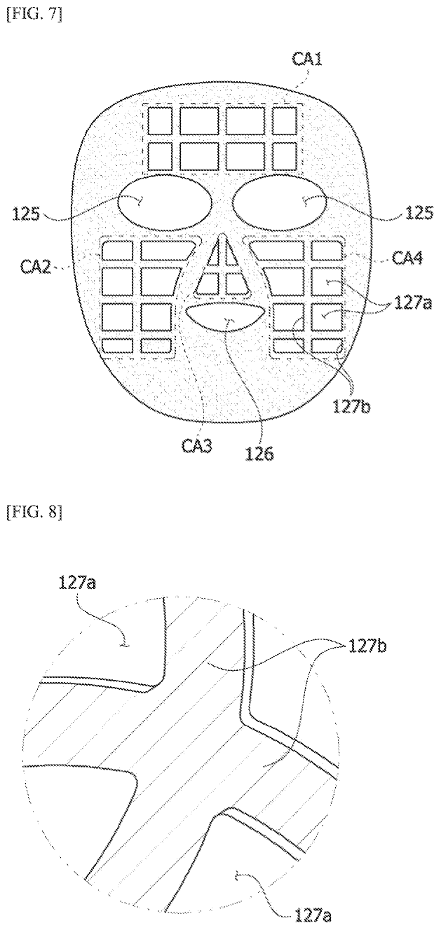

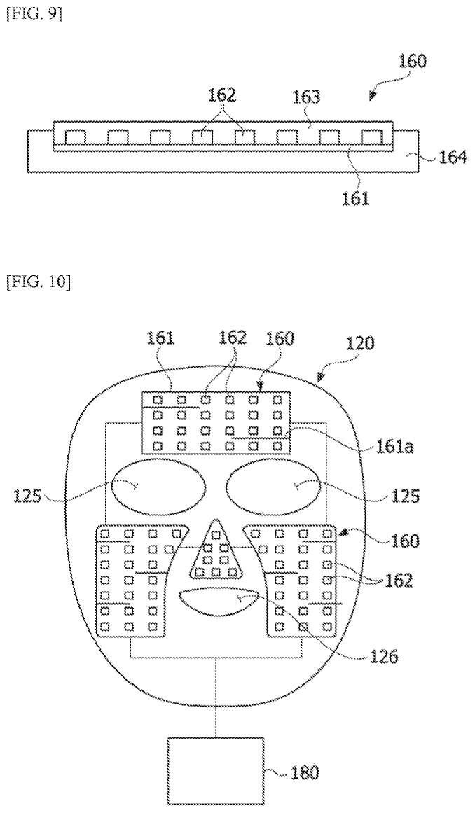

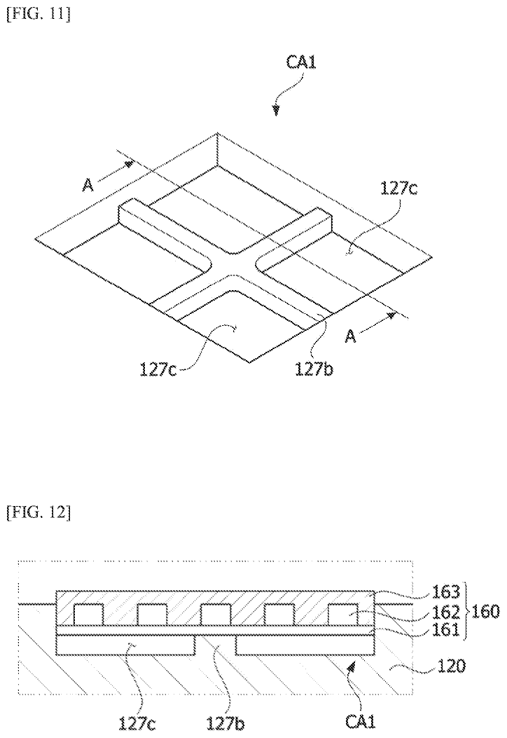

FIG. 7 is a plane view of a cover mask according to the present disclosure, FIG. 8 is an enlarged view of a region in which a light source module is mounted, FIG. 9 is a conceptual diagram of a light source module, FIG. 10 is a plane view showing a state in which a light source module is mounted on a cover mask, FIG. 11 is an enlarged view of a mounting groove formed in a region in which a light source module is mounted, and FIG. 12 shows a state where a light source module is arranged in a mounting groove.

Referring to FIGS. 7 to 9, the cover mask 120 may include a plurality of opening portions 127a formed in regions CA1, CA2, CA3, and CA4 where the light-emitting module 160 is arranged, and a frame portion 127b arranged between the plurality of opening portions 127a.

In the cover mask 120, regions where the light-emitting module 160 is arranged may include regions corresponding to the forehead, cheeks, and the nose, but without being limited thereto, the light-emitting module 160 may be formed in various regions requiring skin care.

The light-emitting module 160 may be attached to a frame portion 127b. The frame portion 127b may be manufactured according to the shape of the face of the user when the cover mask 120 is manufactured, and thus may have a curvature corresponding to the shape of the face of the user. Thus, the light-emitting module 160 attached to the frame portion 127b may also be arranged according to the shape of the face of the user. Moreover, heat generated in the light-emitting module 160 is emitted through the opening portion 127a formed between the frame portions 127b, thereby improving transfer of heat corresponding to the use of the mask to the user.

The light-emitting module 160 may include a lower elastic member 164, the circuit board 161, a plurality of light-emitting elements 162, and an upper elastic pad 163 formed on the circuit board 161 to cover the plurality of light-emitting elements 162. In this case, the lower elastic member and the upper elastic member may be formed integrally by insert injection. When the light-emitting module 160 includes insert molding members 163 and 164, it may be freely bent according to a curvature of the frame portion 127b and attached. A material of the insert molding member is not specially limited. For example, the material of the insert molding member may be the same as the material of the elastic pad.

Referring to FIG. 10, the light-emitting module 160 may be attached to a region where the light-emitting module 160 is arranged, and the plurality of light-emitting modules 160 may be electrically connected. In this case, the plurality of light-emitting modules 160 may be electrically connected to a control/power module 180.

The plurality of light-emitting elements 162 may be arranged in appropriate number and position to irradiate light to the face as a whole. The number and position of the light-emitting elements 162 are not particularly limited.

A slit 161a may be formed in a horizontal direction or a vertical direction in a plurality of circuit boards 161. With this structure, the circuit board 161 may be freely bent according to the shape of the face of the user. Moreover, even when the cover mask 120 is bent due to repeated use, the circuit board 161 may be prevented from being damaged.

Referring to FIGS. 11 and 12, the region CA1 where the light-emitting module 160 is arranged may form the mounting groove 123 inside which the frame portion 127b may be formed. While the frame portion 127b is formed and the other region forms a hole in the above-described structure, a sub-groove 127c may be formed in a peripheral region of the frame portion 127b in the current embodiment.

Based on such a structure, the circuit board 161 and the light-emitting element 162 may be arranged in the mounting groove 123 that may be filled with resin, thus forming the elastic pad 163 as shown in FIG. 1. That is, the circuit board 161 may be bent according to the shape of the face of the user by the frame portion 127b formed in the mounting groove 123, and the elastic pad 163 may be manufactured by filling resin.

In another embodiment, the frame portion 127b may be omitted and the mounting groove may be formed large as one opening portion. In this case, the light-emitting module 160 may be manufactured larger than the opening portion and attached to the inner surface of the cover mask 120.

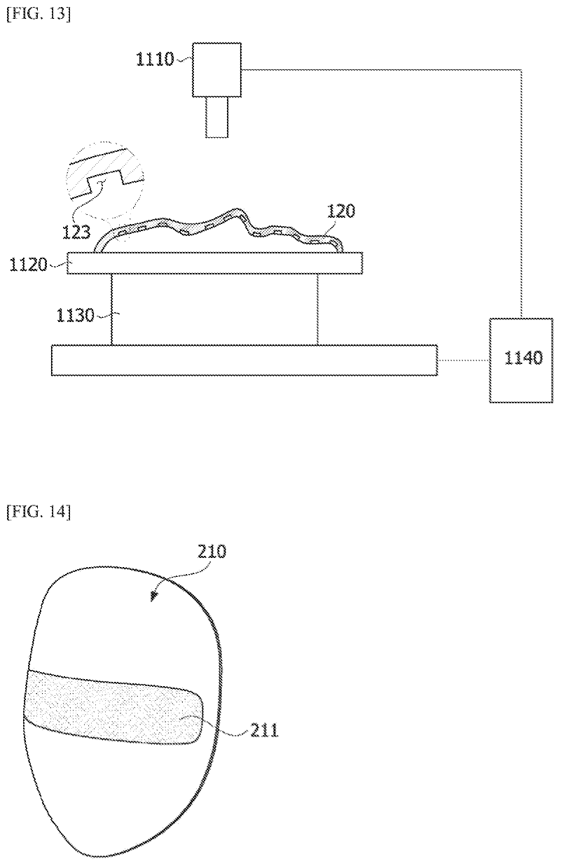

FIG. 13 shows an apparatus for manufacturing a mask.

Referring to FIG. 13, the cover mask 120 and a mold may be manufactured using a 3D printer. For the 3D printer, various any known techniques may be applied.

The 3D printer may manufacture a mask corresponding to the shape of the face while a spraying unit 1110 sprays a printing material onto a stage 1120 in the shape of dots. A controller 1140 may control the spraying unit 1110 or a driving unit 1130 according to input facial shape information. The plurality of mounting grooves 123 may be formed in the inner surface of the cover mask 120.

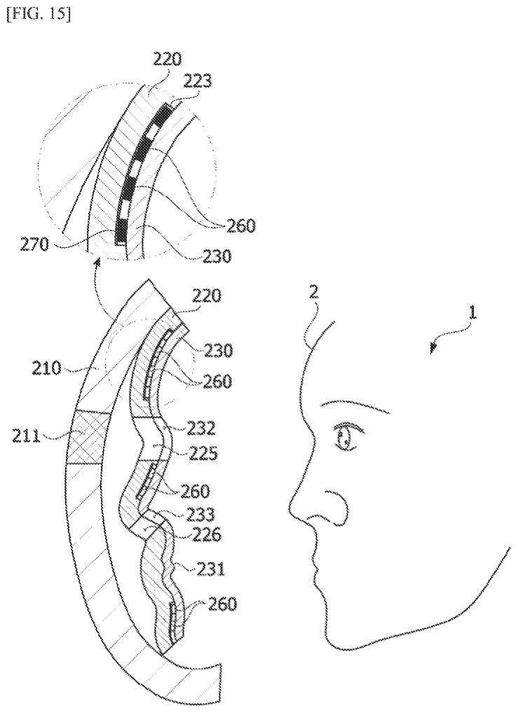

FIG. 14 is a conceptual diagram of a mask module according to a third embodiment of the present disclosure, and FIG. 15 is a conceptual diagram of a mask module according to a third embodiment of the present disclosure.

Referring to FIGS. 14 and 15, a mask module according to an embodiment may include an elastic pad 230 having a first inner surface 231 corresponding to the shape of the face of the user, a cover mask 220 having a second inner surface corresponding to the first inner surface 231, and a plurality of light-emitting elements 260 irradiating light toward the face or the skin of the user.

The elastic pad 230 may have the first inner surface 231 and a first outer surface that correspond to a facial shape 22 of the user 21. The elastic pad 230 may be separately manufactured according to the facial shape of the user. By manufacturing the elastic pad 230 based on facial shape information of the user, the elastic pad 230, when worn, may closely contact the face of the user. Herein, close contact may define that a contact region where a gap with the face of the user is less than or equal to 1 mm is greater than or equal to 70% with respect to 100% of an area of the elastic pad 230.

The elastic pad 230 may be manufactured of an elastic material not to excessively press the face of the user. For example, the elastic pad 230 may be manufactured of, but not limited to, polymer resin such as silicone. A material for the elastic pad 230 may be selected without a limitation when the material is capable of passing light therethrough and has elasticity.

A thickness of the elastic pad 230 may be about 0.1 mm to about 10.0 mm. When the thickness is less than 0.1 mm, the elastic pad 230 may be excessively thin and may be torn when used, and when the thickness is greater than 10.0 mm, pressure may be put on the face due to a weight and it may be difficult to manufacture the elastic pad 230 finely to correspond to the shape of the face.

The elastic pad 230 may include opening portions 232 and 233 that expose the eye and the nose of the user. In addition, although not shown in the drawing, the elastic pad 230 may further include an opening portion that exposes the mouth of the user. However, without being necessarily limited thereto, some opening portion may not be formed when necessary.

The cover mask 220 may be manufactured with, but not necessarily limited to, a plastic material, and various materials having a predetermined strength may also be selected. Both an inner surface and an outer surface of the cover mask 220 may have the same shape as the shape of the face of the user. The cover mask 220 may be separately manufactured using the facial shape information of the user in the same manner as the elastic pad 230.

The cover mask 220 may include opening portions 225 and 226 that expose the eye and the nose of the user. In addition, although not shown in the drawing, the elastic pad 230 may further include an opening portion that exposes the mouth of the user. However, without being necessarily limited thereto, some opening portion may not be formed when necessary.

The user may put the elastic pad 230 on the face and then wear the cover mask 220. That is, the elastic pad 230 may be manufactured individually in a state of being separated from the cover mask 220 without being fixed thereto. A reflection film (not shown) may be coated on the inner surface of the cover mask 220 to reflect light reflected from the skin of the user toward the skin.

The plurality of light-emitting elements 260 may be arranged on the cover mask 220 or the elastic pad 230 to irradiate light toward the first inner surface 231 of the elastic pad 230. Thus, light passing through the elastic pad 230 may be irradiated to the face or the skin of the user. In the inner surface of the cover mask 220, an insertion groove 223 may be formed into which the light-emitting element 260 and a circuit board 270 may be inserted.

The plurality of light-emitting elements 260 may irradiate light in a wavelength band that is useful for the skin. For example, the plurality of light-emitting elements 260 may have an output wavelength of at least one of 390 nm, 450 nm, 580 nm, 630 nm, and 700 nm. The plurality of light-emitting elements 260 may have different output wavelengths. For example, the plurality of light-emitting elements 260 may emit light in a near-infrared wavelength band of 600 nm to 940 nm.

The plurality of light-emitting elements 260 may be light-emitting diodes, organic light-emitting diodes, or laser diodes. That is, the plurality of light-emitting elements are not specially limited as long as they may irradiate light helpful to improve a facial skin tone for brightening the same or maintain facial elasticity.

The case 210 may be coupled to the cover mask 220 to protect an exterior of a mask module. The case 210 may have arranged therein a window 211 through which the user may observe an outside.

The cover mask 220, when the case 210 is attached thereto, may be manufactured to merely have a functional part, thereby simplifying a manufacturing process. When the cover mask 220 is an outermost cover, the cover mask needs to be finished separately.

That is, the elastic pad 230 and the cover mask 220 are manufactured in a customized shape depending on the user, but the case 210 may be manufactured in a standardized single size. The case 210 is manufactured to have a streamlined shape, but the cover mask 220 may be manufactured to correspond to the shape of the face of the user. Thus, a gap may be formed between the case 210 and the cover mask 220.

The case 210 or the cover mask 220 may accommodate required parts of a mask module, e.g., a control module, a circuit module, a power module, etc.

A fixing unit (not shown) connected to the cover mask 220 or the case 210 to fix the mask module to the head of the user may be further included. The fixing unit may be of a band type, but may not be particularly limited as long as it is capable of fixing the cover mask 220 or the case 210 to the head of the user by being connected thereto.

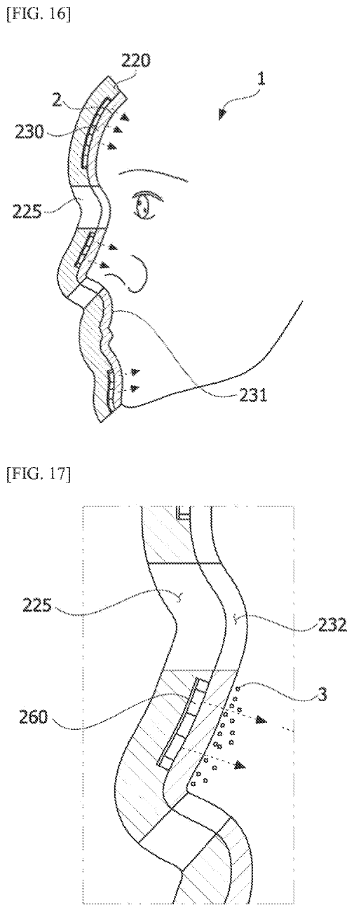

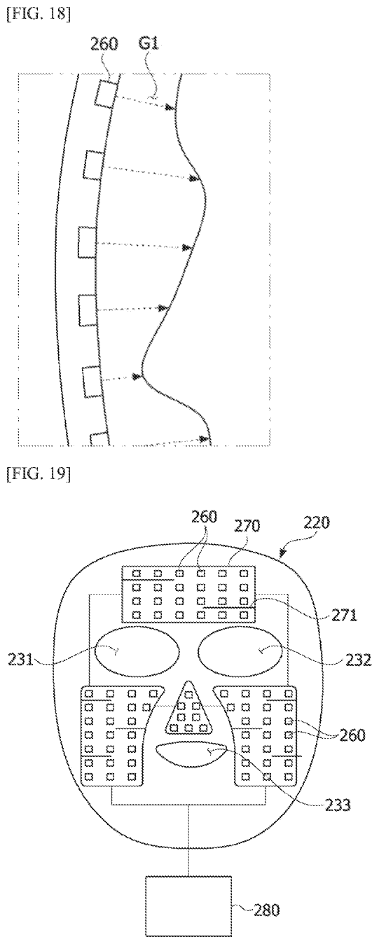

FIG. 16 is a conceptual diagram of a mask module according to a fourth embodiment of the present disclosure, FIG. 17 shows that an elastic pad is in close contact with a face to prevent moisture from being evaporated, and FIG. 18 shows a state in which sweat is evaporated through a gap between a conventional LED mask and a face.

Referring to FIGS. 16 and 17, a cover may be omitted from the mask module according to an embodiment. That is, the mask module may include the elastic pad 230 and the cover mask 220. In this case, required parts of the mask module, e.g., a control module, a circuit module, a power module, etc., may be connected to the cover mask 220.

According to an embodiment, the inner surface and the outer surface of the cover mask 220 have a shape corresponding to the shape of the face 2 of the user 1, such that the light-emitting element 260 mounted on the cover mask 220 may also be arranged according to the shape of the face of the user. Thus, light may be irradiated substantially perpendicularly to the face of the user. As a result, light injection efficiency may be improved by minimizing light reflection from the skin.

According to an embodiment, the elastic pad 230 closely contacts the face, such that moisture 3 such as sweat generated by light irradiation may not be discharged to the outside, thus maintaining a moisturizing effect. Moreover, as the skin does become dry, a beauty effect may be maximized by sufficiently increasing the intensity of light.

Referring to FIG. 18, the gap G1 exists between the inner surface of the cover mask 220 and the face in the conventional LED mask, such that moisture (sweat) and heat generated by light irradiation are easily discharged to the outside, making the skin dry. When the power of light is increased in a dry state of the skin, the skin may be even damaged. Moreover, LEDs are not arranged along the face, such that light may not be incident perpendicularly, lowering injection efficiency.

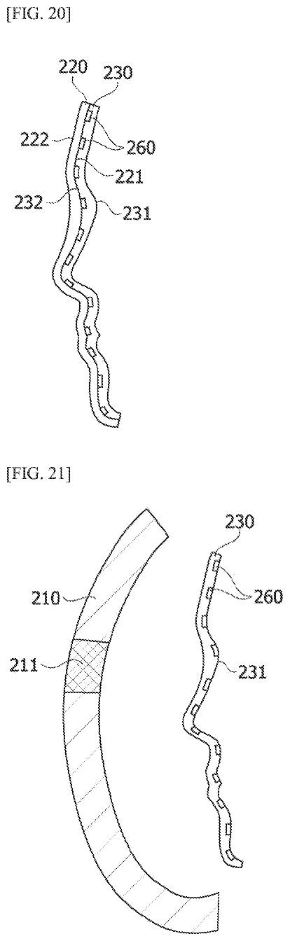

FIG. 19 shows arrangement of a plurality of light-emitting elements, FIG. 20 shows a mask module according to a fifth embodiment of the present disclosure, and FIG. 21 shows a mask module according to a sixth embodiment of the present disclosure.

Referring to FIG. 19, the plurality of light-emitting elements 260 may be arranged in appropriate number and position to irradiate light to the face as a whole. The number and position of the light-emitting elements 260 are not particularly limited.

The plurality of circuit boards 270 may be arranged on the inner surface of the cover mask 220. A slit 271 may be formed in a horizontal direction or a vertical direction in the plurality of circuit boards 270. With this structure, the circuit board 270 may be freely bent according to the shape of the face of the user. Moreover, even when the cover mask 220 or the elastic pad 230 is bent due to repeated use, the circuit board 270 may be prevented from being damaged.

The number and shape of the plurality of circuit boards 270 are not particularly limited. That is, the number of circuit boards 270, the shape of the circuit boards 270, and the number of light-emitting elements 260 may be appropriately adjusted to irradiate light uniformly to the face of the user. The plurality of circuit boards 270 may be connected to a power/control module 280.

Although it is described that the plurality of light-emitting elements 260 are arranged in the insertion groove formed in the cover mask 220, the present disclosure is not limited thereto. When the elastic pad 230 is sufficiently thick as shown in FIG. 20, the light-emitting element may be arranged on the inner surface or the outer surface 232. In this case, the mask module may include the elastic pad 230 without the cover mask.

Referring to FIG. 21, the light-emitting element 260 may be arranged inside the elastic pad 230 and the exterior may be formed with the case 210. That is, the user may put the elastic pad 230 on the face and then wear the case 210. However, without being necessarily limited thereto, the user may place and use the elastic pad 230 without mounting the case 210.

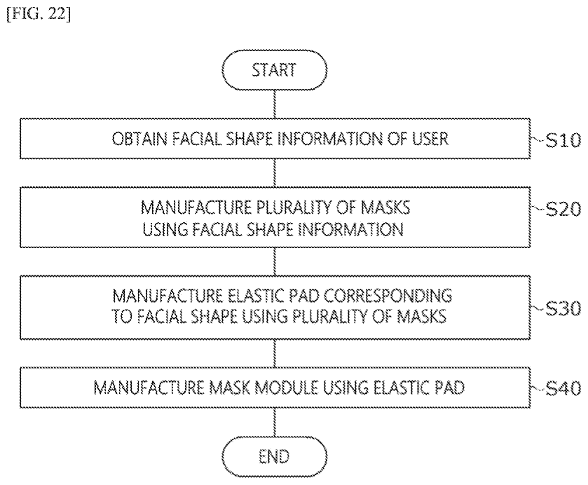

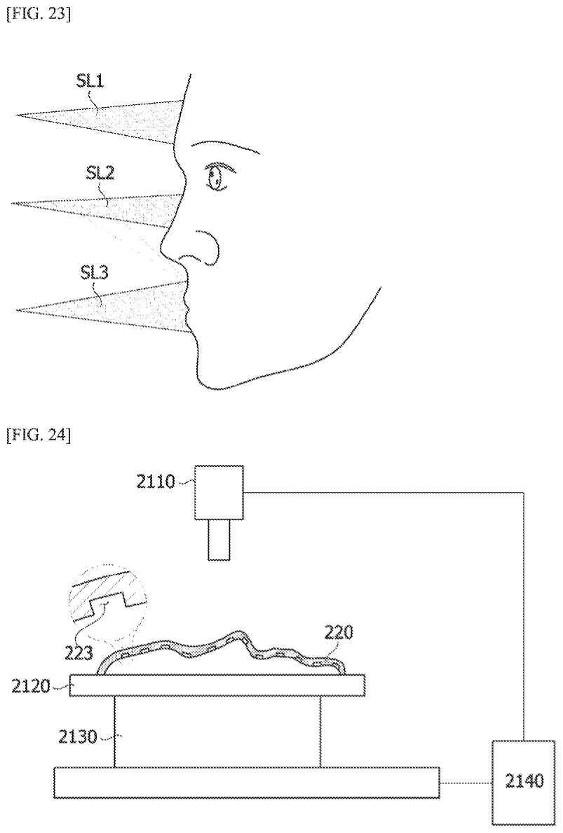

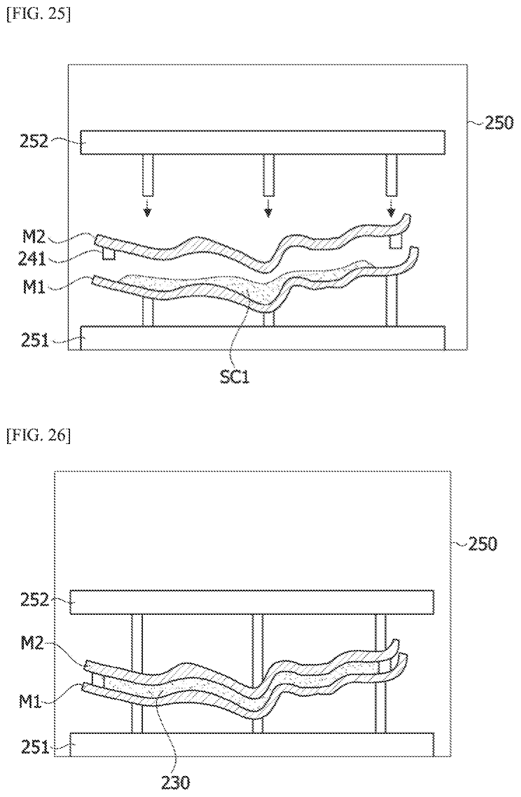

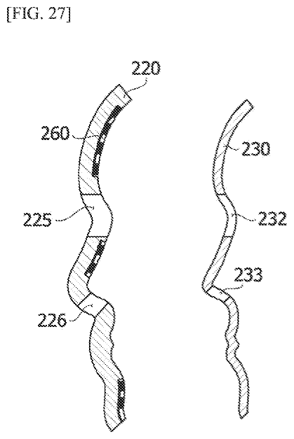

FIG. 22 is a flowchart of a method for manufacturing a mask module, according to a third embodiment of the present disclosure, FIG. 23 shows a process of obtaining facial shape information, FIG. 24 shows an apparatus for manufacturing a mask, FIGS. 25 and 26 show a process of manufacturing an elastic pad by using a mold, and FIG. 27 is a conceptual diagram of a manufactured mask module.

Referring to FIG. 22, a method for manufacturing a mask module according to an embodiment may include operation S10 of obtaining facial shape information of a user, operation S20 of manufacturing a plurality of masks using the facial shape information, operation S30 of manufacturing an elastic pad, and operation S40 of assembling the mask module using the elastic pad.

Referring to FIG. 23, operation S10 of obtaining the facial shape information of the user may include obtaining the facial shape information of the user who is to use the mask module. The facial shape information of the user may be obtained using, but not necessarily limited to, a measurement apparatus such as, for example, laser scanners SL1, SL2, and SL3.

By way of example, when the user downloads and runs an application (App) through a terminal such as a smartphone owned by the user, the application may be driven such that the terminal may operate in a scan mode. When the user scans the face in a way instructed by the application, the facial shape information may be stored in the terminal and transmitted to a server.

However, without being limited thereto, the method may be limitlessly applied to a region requiring skin massage such as a hand, a neck, etc., as well as the face of the user. In particular, when the light-emitting element is arranged on the elastic pad, the mask apparatus may be worn limitlessly on various body parts. Hereinbelow, description will be made based on wearing on the face of the user by way of example.

Operation S20 of manufacturing the plurality of masks may include manufacturing a mold for manufacturing the cover mask 220 and the elastic pad 230 that constitute the mask module.

Referring to FIG. 24, the cover mask 220 and the mold may be manufactured using a 3D printer. For the 3D printer, various any known techniques may be applied.

The 3D printer may manufacture a mask corresponding to the shape of the face while a spraying unit 2110 sprays a printing material onto a stage 2120 in the shape of dots. A controller 2140 may control the spraying unit 2110 or a driving unit 2130 according to input facial shape information.

A total of three masks may be manufactured. One of them may be the cover mask 220 used as the mask module, and the other two of them may be molds for manufacturing the elastic pad. The cover mask 220 may have formed therein an opening portion to secure a user's view and allow breathing. The opening portion may be formed by manufacturing and processing the cover mask 220, and may be formed in an operation of manufacturing the cover mask 220.

The mold may not form the opening portion to prevent liquid silicone from flowing. However, without being necessarily limited thereto, the cover mask 220 used as the mask module may also be used as the mold. In this case, the opening portion formed in the cover mask 220 may be temporarily closed.

Referring to FIGS. 25 and 26, operation S30 of manufacturing the elastic pad 230 may include arranging a first mold M1 and a second mold M2, manufactured using a 3D printer, on a nest plate 251 of a chamber 250 separately in a vertical direction and filling liquid silicone SC1 therebetween.

Thereafter, the elastic pad 230 may be manufactured by pressing the second mold M2 by using a pressing member 252. In this case, the elastic pad 230 may have a predetermined thickness as the first mold M1 and the second mold M2 are separated from each other by a height of a protrusion pin 241. A heater for heating the first mold M1 and the second mold M2 may be further provided to spread the liquid silicone well, but the present disclosure is not limited thereto.

Referring to FIG. 27, operation S40 of assembling the mask module may include fixing a circuit board on which the plurality of light-emitting elements 260 are arranged to the cover mask 220 and connecting a power module, etc., for applying power to the circuit board. Opening portions may be formed in positions where the eye, the nose, and the mouth of the user are arranged on the cover mask 220 and the elastic pad 230.

The user may put the elastic pad 230 on the face first and then wear the cover mask 220 for use. However, without being limited thereto, when the light-emitting element 260 is mounted on the elastic pad 230, the elastic pad 230 may be put on the face first and then the case may be worn so as to be used. Alternatively, the elastic pad 230 may be put on the face so as to be used, without the cover mask 220 and the case.

While the embodiments have been described, they are merely examples and do not limit the present disclosure, and it would be understood by those of ordinary skill in the art that several modifications and applications not described above are possible without departing the essential characteristics of the current embodiment. For example, each component described in detail in the embodiment may be carried out by being modified. Differences related to such modifications and applications should be interpreted as falling within the scope of the present invention defined in the appended claims.

1. A mask apparatus comprising:
a cover mask having a curvature corresponding to a shape of a face of a user; and
a plurality of light-emitting modules arranged in the cover mask to irradiate light to the face of the user.

2. The mask apparatus of claim 1, wherein the cover mask comprises a plurality of mounting grooves mounted on an inner surface thereof,
the plurality of light-emitting modules are arranged in the plurality of mounting grooves, and
the light-emitting module comprises:
a circuit board arranged in each of the plurality of grooves; and
a plurality of light-emitting elements arranged in the circuit board.

3. The mask apparatus of claim 2, wherein the light-emitting module comprises an elastic pad filled in the mounting groove to cover the light-emitting element, and
the elastic pad protrudes from the inner surface of the cover mask such that a top surface of the elastic pad contacts the face of the user.

4. The mask apparatus of claim 2, further comprising a cover film formed in the inner surface of the cover mask to cover the mounting groove.

5. The mask apparatus of claim 1, wherein the cover mask comprises a plurality of opening portions formed in a region in which the light-emitting module is arranged, and a frame portion arranged between the plurality of opening portions, and
the light-emitting module is attached to the frame portion.

6. The mask apparatus of claim 5, wherein the light-emitting module comprises a circuit board, a plurality of light-emitting elements arranged on the circuit board, and an insert molding member covering the circuit board and the plurality of light-emitting elements, and
a surface of the insert molding member is attached to the frame portion.

7. The mask apparatus of claim 1, wherein the region in which the light-emitting module is arranged comprises a region corresponding to a forehead, a cheek, and a nose of the user.

8. The mask apparatus of claim 5, wherein the frame portion has the curvature corresponding to the shape of the face of the user.

9. The mask apparatus of claim 1, further comprising a case arranged on an outer side of the cover mask.
